(19) United States
(12) Patent Application Publication
(10) Pub. No.: US 2017/0049379 A1
(43) Pub. Date: Feb. 23, 2017

(54) MRI CHARACTERIZATION OF PLACENTAL OXYGEN TRANSPORT

(71) Applicants: MASSACHUSETTS INSTITUTE OF TECHNOLOGY, Cambridge, MA (US); CHILDREN'S MEDICAL CENTER CORPORATION, Boston, MA (US); UNIVERSIDAD REY JUAN CARLOS, Madrid (ES)

(72) Inventors: Jie Luo, Cambridge, MA (US); Esra Abaci Turk, Boston, MA (US); Patricia Ellen Grant, Newton, MA (US); Norberto Malpica, Madrid (ES); Elfar ADALSTEINSSON, Belmont, MA (US)

(21) Appl. No.: 15/137,859

(22) Filed: Apr. 25, 2016

Related U.S. Application Data
(60) Provisional application No. 62/152,125, filed on Apr. 24, 2015.

(51) Int. Cl. A61B 5/00; A61B 5/145; G06T 7/00; G01R 33/561; G01R 33/565; G06K 9/46; A61B 5/055; G01R 33/483

(52) U.S. Cl. CPC A61B 5/4362; A61B 5/055; A61B 5/14542; A61B 5/7282; A61B 5/742; A61B 5/7485; G01R 33/4833; G01R 33/5616; G01R 33/56509; G06K 9/4604; G06T 7/0036; A61B 2576/00; G06T 2207/10088

(57) ABSTRACT

A method is provided for generating images using a MRI system. The method includes one or more acts below. First, the MRI system applies a pulse sequence to obtain a first set of blood oxygenation level dependent (BOLD) MRI images of a pregnant subject during a first time period. The MRI system then applies the pulse sequence to obtain a second set of BOLD MRI images of the pregnant subject during a second time period. The MRI system automatically extracts one or more regions of interest that include a placenta of the pregnant subject in the first and second sets of BOLD MRI images. The MRI system obtains BOLD signal changes in the one or more regions of interest based on the first and second sets of BOLD MRI images. The MRI system generates, based on the BOLD signal changes, a map indicating placental oxygen transport.

### CROSS-REFERENCE TO RELATED APPLICATIONS

This application claims the benefit of U.S. Provisional Patent Application Ser. No. 62/152,125, filed on Apr. 24, 2015, and entitled “MRI Characterization of Placental Oxygen Transport,” which is incorporated herein by reference in its entirety.

### STATEMENT OF FEDERALLY SPONSORED RESEARCH OR DEVELOPMENT

This invention was made with government support under EB017337 and HD087211 awarded by the National Institutes of Health. The government has certain rights in the invention.

### BACKGROUND

Oxygen transport across the placenta is critically dependent on blood flow in the uterine and umbilical vessels, as well as blood oxygen content and the diffusing capacity of the placenta. Clinically, Doppler ultrasound is used to measure umbilical artery blood flow as a surrogate of blood perfusion without direct measurement of oxygen delivery. There is a need to develop an imaging method to direct measure of oxygen delivery.

### SUMMARY

In the description, reference is made to the accompanying drawings that form a part hereof, and in which examples of the invention are provided. Such examples do not necessarily represent the full scope of the invention, however, and reference is made therefore to the claims and herein for interpreting the scope of the invention.

In a first aspect, a method is provided for generating images using a MRI system. The method includes one or more acts below. First, the MRI system applies a pulse sequence to obtain a first set of blood oxygenation level dependent (BOLD) MRI images of a pregnant subject during a first time period. The MRI system then applies the pulse sequence to obtain a second set of BOLD MRI images of the pregnant subject during a second time period. The MRI system automatically extracts one or more regions of interest that include a placenta of the pregnant subject in the first and second sets of BOLD MRI images. The MRI system obtains BOLD signal changes in the one or more regions of interest based on the first and second sets of BOLD MRI images. The MRI system generates, based on the BOLD signal changes, a map indicating placental oxygen transport.

In a second aspect, a MRI system includes a magnet system configured to generate a static magnetic field about at least a placenta of a subject arranged in the MRI system. The MRI system includes at least one gradient coil configured to establish at least one magnetic gradient field with respect to the static magnetic field. The MRI system includes a radio frequency (RF) system configured to deliver excitation pulses to the subject. The MRI system also includes a computer system. The computer system is programmed to: control the at least one gradient coil and the RF system to perform a pulse sequence to obtain a first set of blood oxygenation level dependent (BOLD) MRI images on the subject during a first time period; control the at least one gradient coil and the RF system to perform the pulse sequence to obtain a second set of BOLD MRI images on the subject during a second time period; automatically extract one or more regions of interest in the first and second sets of BOLD MRI images; obtain BOLD signal changes in the one or more regions of interest based on the first and second sets of BOLD MRI images; and generate, based on the BOLD signal changes, a map indicating placental oxygen transport.

### BRIEF DESCRIPTION OF THE DRAWINGS

FIG. 1 is a block diagram of an example magnetic resonance imaging (“MRI”) system configured for use in accordance with the present disclosure.

FIG. 2 is a flowchart setting forth steps of an example method according to the disclosure.

FIG. 3 is a flowchart illustrating additional steps of the example method according to the disclosure.

FIG. 4 is a flowchart illustrating additional steps of the example method according to the disclosure.

FIG. 5 is a flowchart illustrating additional steps of the example method according to the disclosure.

FIG. 6 is a flowchart illustrating steps of an example method to estimate and control signal non-uniformities and motion artifacts in the MRI images according to the disclosure.

FIG. 7 is a flowchart illustrating steps of an example method using N4 Bias correction on the MRI images.

FIG. 8a is a flowchart illustrating steps for motion correction within a volume using group-wise registration.

FIG. 8b is a flowchart illustrating steps for motion correction between volumes using pair-wise registration.

FIG. 9 is a flowchart illustrating steps of outlier rejection after motion correction.

FIG. 10 is an example of segmentation of placenta (red), fetal brains (light green and dark green) and fetal livers (light brown and dark brown).

FIG. 11a shows volume overlap computed before applying the pipeline (blue), after applying the pipeline (red), and after outlier voxel rejection (green) for placenta region.

FIG. 11b shows volume overlap computed before applying the pipeline (blue), after applying the pipeline (red), and after outlier voxel rejection (green) for liver region.

FIG. 11c shows volume overlap computed before applying the pipeline (blue), after applying the pipeline (red), and after outlier voxel rejection (green) for brain region. The error bars are computed across volumes in the time series.

FIG. 11d shows comparison of combined Dice coefficients with the error bars computed across all subjects and all volumes for each subject.

FIG. 12a shows intermediate images after each processing step in the pipeline for two subjects.

FIG. 12b shows intensity vs. time for the fetal liver of the second subject.

FIG. 12c shows intensity vs. time for the fetal liver of the third subject.

FIG. 13a shows overall percentages of rejected volumes in the analysis of different regions in the fetus.

FIG. 13b shows percentage of rejected volumes in the analyses of the different regions after each step of outlier detection.

FIG. 14 shows example images illustrating effects of motion correction within the volume to the alignment of the volumes in a reference volume.

FIG. 15 shows an example signal change curve with two delay parameters to be estimated.

FIG. 16a shows examples images illustrating T2* map and an estimated first delay parameter map superimposed on the MRI images.

FIG. 16b shows examples images illustrating an estimated second delay parameter map superimposed on the MRI images and histograms.

FIG. 17a shows examples illustrating grouped time activity curves of ΔR2* for placenta.

FIG. 17b shows examples illustrating grouped time activity curves of ΔR2* for fetal brains.

FIG. 17c shows examples illustrating grouped time activity curves of ΔR2* for fetal livers.

FIG. 18 shows a table illustrating individual analysis of time activity curves (TAC) of each fetus.

FIG. 19a shows example images illustrating segmentation of placenta and fetal organs in twins separately.

FIG. 19b shows example images illustrating segmentation of placenta and fetal organs in twins together.

### DETAILED DESCRIPTION OF THE INVENTION

Blood oxygenation level dependent (BOLD) imaging is the standard technique used to generate images in functional MRI (fMRI) studies. fMRI relies on the BOLD contrast mechanism, which derives contrast from the magnetic properties of hemoglobin. Deoxyhemoglobin is paramagnetic in contrast to oxyhemogolobin, which is diamagnetic. During maternal hyperoxia, the concentration of deoxyhemoglobin decrease and the local magnetic field changes thereby increasing the T2* relaxation time of the neighboring protons, which produces a measurable increase in the local BOLD MRI signal.

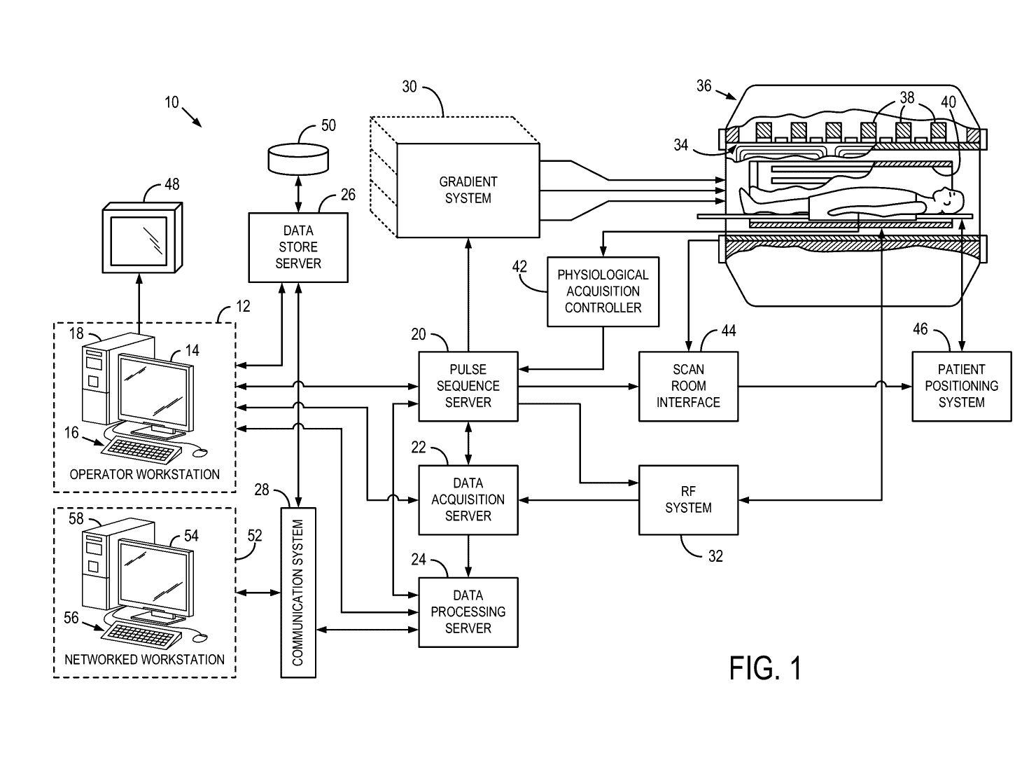

Referring particularly now to FIG. 1, an example of a magnetic resonance imaging (MRI) system 10 that can implement the methods described here is illustrated. The MRI system 10 includes an operator workstation 12 that may include a display 14, one or more input devices 16 (e.g., a keyboard, a mouse), and a processor 18. The processor 18 may include a commercially available programmable machine running a commercially available operating system. The operator workstation 12 provides an operator interface that facilitates entering scan parameters into the MRI system 10. The operator workstation 12 may be coupled to different servers, including, for example, a pulse sequence server 210, a data acquisition server 212, a data processing server 214, and a data store server 216. The operator workstation 12 and the servers 20, 22, 24, and 26 may be connected via a communication system 28, which may include wired or wireless network connections.

The pulse sequence server 20 functions in response to instructions provided by the operator workstation 12 to operate a gradient system 30 and a radiofrequency (“RF”) system 32. Gradient waveforms for performing a prescribed scan are produced and applied to the gradient system 30, which then excites gradient coils in an assembly 34 to produce the magnetic field gradients Gx, Gy, and Gz that are used for spatially encoding magnetic resonance signals. The gradient coil assembly 34 forms part of a magnet assembly 36 that includes a polarizing magnet 38 and a whole-body RF coil 40.

RF waveforms are applied by the RF system 32 to the RF coil 40, or a separate local coil to perform the prescribed magnetic resonance pulse sequence. Responsive magnetic resonance signals detected by the RF coil 40, or a separate local coil, are received by the RF system 32. The responsive magnetic resonance signals may be amplified, demodulated, filtered, and digitized under direction of commands produced by the pulse sequence server 20. The RF system 32 includes an RF transmitter for producing a wide variety of RF pulses used in MRI pulse sequences. The RF transmitter is responsive to the prescribed scan and direction from the pulse sequence server 20 to produce RF pulses of the desired frequency, phase, and pulse amplitude waveform. The generated RF pulses may be applied to the whole-body RF coil 40 or to one or more local coils or coil arrays.

The RF system 32 also includes one or more RF receiver channels. An RF receiver channel includes an RF preamplifier that amplifies the magnetic resonance signal received by the coil 40 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²} (1);

and the phase of the received magnetic resonance signal may also be determined according to the following relationship:

$$ϕ = {tan}^{- 1} ( \frac{Q}{I} ) . \qquad ( 2 )$$

The pulse sequence server 20 may receive patient data from a physiological acquisition controller 42. By way of example, the physiological acquisition controller 42 may receive signals from a number of different sensors connected to the patient, including electrocardiograph (“ECG”) signals from electrodes, or respiratory signals from a respiratory bellows or other respiratory monitoring devices. These signals may be used by the pulse sequence server 20 to synchronize, or “gate,” the performance of the scan with the subject's heart beat or respiration. Also, as will be described, a non-rebreather facial mask 43 may be used, which may be connected, for example, to an oxygen supply flowmeter on the wall of the scanner room.

The pulse sequence server 20 may also connect to a scan room interface circuit 232 that receives signals from various sensors associated with the condition of the patient and the magnet system. Through the scan room interface circuit 44, a patient positioning system 46 can receive commands to move the patient to desired positions during the scan.

The digitized magnetic resonance signal samples produced by the RF system 32 are received by the data acquisition server 22. The data acquisition server 22 operates in response to instructions downloaded from the operator workstation 12 to receive the real-time magnetic resonance data and provide buffer storage, so that data is not lost by data overrun. In some scans, the data acquisition server 22 passes the acquired magnetic resonance data to the data processor server 24. In scans that require information derived from acquired magnetic resonance data to control the further performance of the scan, the data acquisition server 22 may be programmed to produce such information and convey it to the pulse sequence server 20. For example, during pre-scans, magnetic resonance data may be acquired and used to calibrate the pulse sequence performed by the pulse sequence server 20. As another example, navigator signals may be acquired and used to adjust the operating parameters of the RF system 32 or the gradient system 30, or to control the view order in which k-space is sampled. In still another example, the data acquisition server 22 may also process magnetic resonance signals used to detect the arrival of a contrast agent in a magnetic resonance angiography (“MRA”) scan. For example, the data acquisition server 22 may acquire magnetic resonance data and processes it in real-time to produce information that is used to control the scan.

The data processing server 24 receives magnetic resonance data from the data acquisition server 22 and processes the magnetic resonance data in accordance with instructions provided by the operator workstation 12. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 24 are conveyed back to the operator workstation 12 for storage. Real-time images may be stored in a data base memory cache, from which they may be output to operator display 14 or a separate display 48. Batch mode images or selected real time images may be stored in a host database on disc storage 50. When such images have been reconstructed and transferred to storage, the data processing server 24 may notify the data store server 26 on the operator workstation 12. The operator workstation 12 may be used by an operator to archive the images, produce films, or send the images via a network to other facilities.

The MRI system 10 may also include one or more networked workstations 52. For example, a networked workstation 52 may include a display 54, one or more input devices 56 (e.g., a keyboard, a mouse), and a processor 58. The networked workstation 52 may be located within the same facility as the operator workstation 12, or in a different facility, such as a different healthcare institution or clinic.

The networked workstation 52 may gain remote access to the data processing server 24 or data store server 26 via the communication system 28. Accordingly, multiple networked workstations 52 may have access to the data processing server 24 and the data store server 26. In this manner, magnetic resonance data, reconstructed images, or other data may be exchanged between the data processing server 24 or the data store server 26 and the networked workstations 52, such that the data or images may be remotely processed by a networked workstation 52.

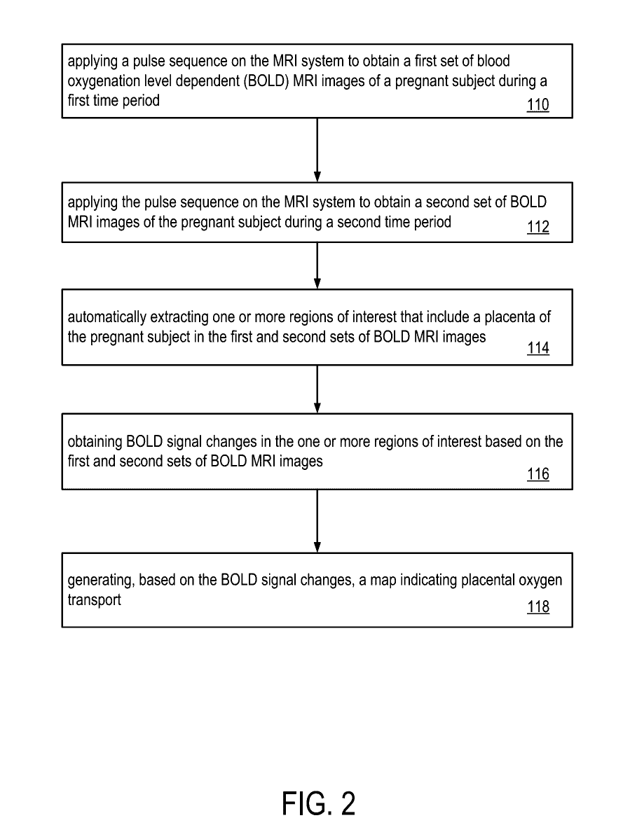

FIG. 2 is a flowchart setting forth the steps of an example method according to the disclosure. The example method may be implemented using a MRI system illustrated in FIG. 1. In FIG. 2, the MRI system may perform the following acts to obtain a map indicating placental oxygen transport.

In step 110, an MRI system, such as described above with respect to FIG. 1, applies a pulse sequence on the MRI system to obtain a first set of blood oxygenation level dependent (BOLD) MRI images of a pregnant subject during a first time period. In step 112, the MRI system applies the pulse sequence on the MRI system to obtain a second set of BOLD MRI images of the pregnant subject during a second time period. The first and second time period may last, as a non-limiting example, about 5 to 10 minutes. For example, the first time period may last 5 minutes and the second time period may last 10 minutes. Alternatively, the first and the second time period may both last 10 minutes.

In step 114, the MRI system automatically align one or more regions of interest that include a placenta of the pregnant subject in the first and second sets of BOLD MRI images. In step 116, the MRI system obtains BOLD signal changes in the one or more regions of interest based on the first and second sets of BOLD MRI images. In step 118, the MRI system generates, based on the BOLD signal changes, a map indicating placental oxygen transport. The map may include a placental gradient map generated from linear fitting of time points within the first preset time period of hyperoxia. As one non-limiting example, the first preset time period may be one minute, two minutes, three minutes, or the like. A 5×5×5 smoothing filter may be applied to control motion artifacts. In the placenta, the gradient map revealed distinct regions of response to oxygen challenge, which were not apparent on the T2* images.

Regions in the placenta, such as regions of ˜3 cm³ in size, may be selected in 3D according to these gradient maps. The delay time of BOLD signal increase may be a useful metric in fetal monitoring. Considering the percent signal increase in brain vessels versus the whole brain, it is likely that their difference owes largely to the low vascular density in the brain parenchyma. The small percent increase in brain parenchymal with hyperoxia brings into question the ability to detect smaller regional resting state fluctuations at this gestational age.

The map may include other timing delay parameters estimated using other models in accordance with the present disclosure. For example, the timing delay parameters may be delays that characterize the signal rising time from normoxia to hyperoxia. The timing delay parameters may be delays that characterize the beginning of signal increase and the end of the normoxia period.

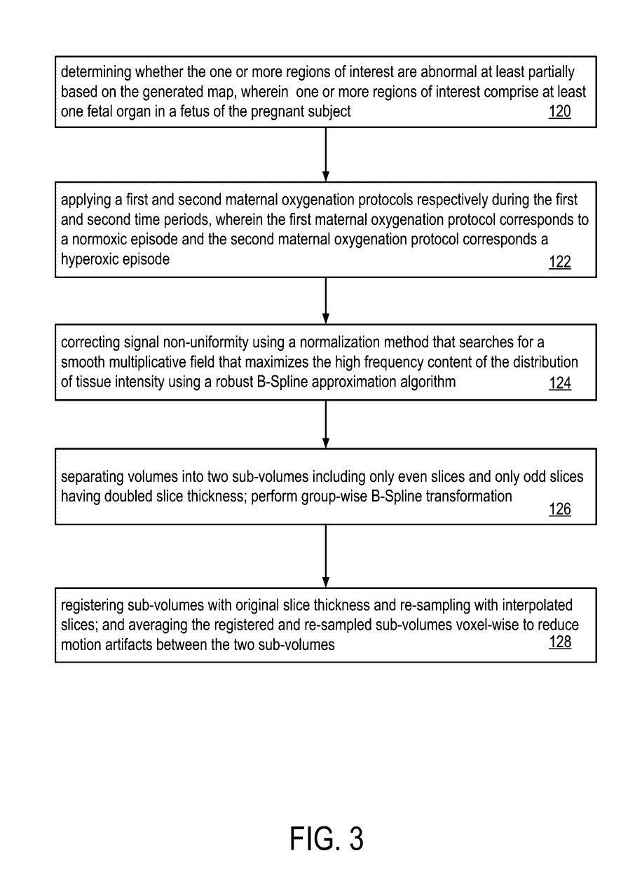

FIG. 3 is a flowchart illustrating additional steps of the example method according to the disclosure. The steps in FIG. 3 may be combined with the steps in FIG. 2.

In step 120, the MRI system determines whether the one or more regions of interest are abnormal at least partially based on the generated map. Here, the one or more regions of interest may include at least one fetal organ in a fetus of the pregnant subject. For example, the regions of interest may include the fetus brain, the fetus liver, or other organs of the fetus.

In step 122, the system applies a first and second maternal oxygenation protocols respectively during the first and second time periods, wherein the first maternal oxygenation protocol corresponds to a normoxic episode and the second maternal oxygenation protocol corresponds a hyperoxic episode. For example, the oxygenation protocol may be applied using a non-rebreather facial mask connected to an oxygen supply flowmeter on the wall of the scanner room (oxygen tank in a separate room). With this configuration, maternal inspired oxygen content (FiO2) was modulated during the MRI scan, as a non-limiting example, from 21% to 100%, to give maternal hyperoxia (FiO2>21%). In step 124, the MRI system corrects signal non-uniformity using a normalization method that searches for a smooth multiplicative field that maximizes the high frequency content of the distribution of tissue intensity using a robust B-Spline approximation algorithm. In step 126, the MRI system separates volumes into two sub-volumes including only even slices and only odd slices having doubled slice thickness. At step 127, the MRI system also registers two sub-volumes using group-wise B-Spline transformation. In step 128, the MRI system re-samples the registered volumes with interpolated slices and averages the registered and re-sampled sub-volume in a voxel-wise fashion to reduce motion artifacts between the two sub-volumes.

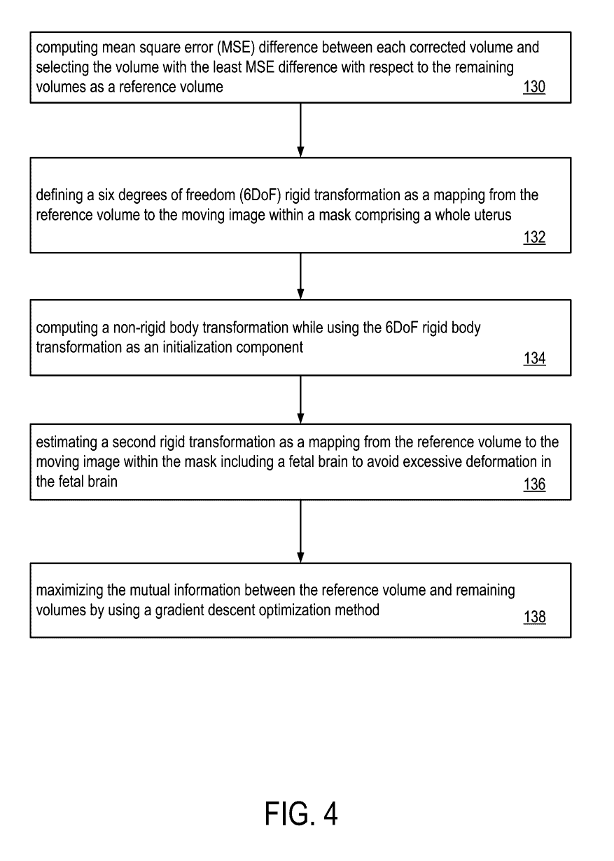

FIG. 4 is a flowchart illustrating additional steps of the example method according to the disclosure. The steps in FIG. 3 may be combined with the steps in FIGS. 2-3.

In step 130, the MRI system may compute mean square error (MSE) difference between each corrected volume and selecting the volume with the least MSE difference with respect to the remaining volumes as a reference volume. In step 132, the MRI system defines a six degrees of freedom (6DoF) rigid transformation as a mapping from the reference volume to the moving image within a mask comprising a whole uterus. In step 134, the MRI system computes a non-rigid body transformation while using the 6DoF rigid body transformation as an initialization component. In step 136, the MRI system estimates a second rigid transformation as a mapping from the reference volume to the moving image within the mask including a fetal brain to avoid excessive deformation in the fetal brain.

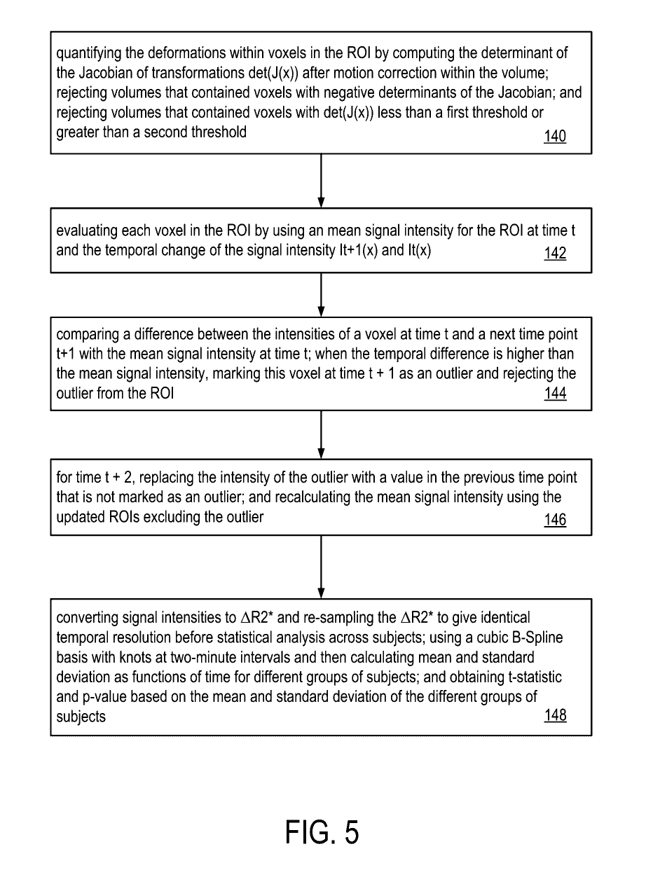

FIG. 5 is a flowchart illustrating additional steps of the example method according to the disclosure. The steps in FIG. 5 may be combined with the steps in FIGS. 2-4.

In step 140, the MRI system quantifies the deformations within voxels in a region of interest (ROI) by computing the determinant of the Jacobian of transformations det(J(x)) after motion correction within the volume; rejects volumes that contained voxels with negative determinants of the Jacobian; and rejects volumes that contained voxels with det(J(x)) less than a first threshold or greater than a second threshold. The first and second thresholds may be determined using previously collected clinical experimental data and other information database.

In step 142, the MRI system evaluating each voxel in the ROI by using a mean signal intensity for the ROI at time t and the temporal change of the signal intensity Iₜ₊₁(x) and Iₜ(x). In step 144, the MRI system compares a difference between the intensities of a voxel at time t and a next time point t+1 with the mean signal intensity at time t. When the temporal difference is higher than the mean signal intensity, the MRI system marks this voxel at time t+1 as an outlier and rejecting the outlier from the ROI. For time t+2, in step 146, the MRI system replaces the intensity of the outlier with a value in the previous time point that is not marked as an outlier. The MRI system then recalculates the mean signal intensity using the updated ROIs excluding the outlier.

In step 148, the MRI system converts signal intensities to ΔR2* and re-sampling the ΔR2* to give identical temporal resolution before statistical analysis across subjects. The MRI system may use a cubic B-Spline basis with knots at two-minute intervals and then calculating mean and standard deviation as functions of time for different groups of subjects. The MRI system may obtain t-statistic and p-value based on the mean and standard deviation of the different groups of subjects. Alternatively or additionally, other statistic model and analysis methods may be used by the MRI system.

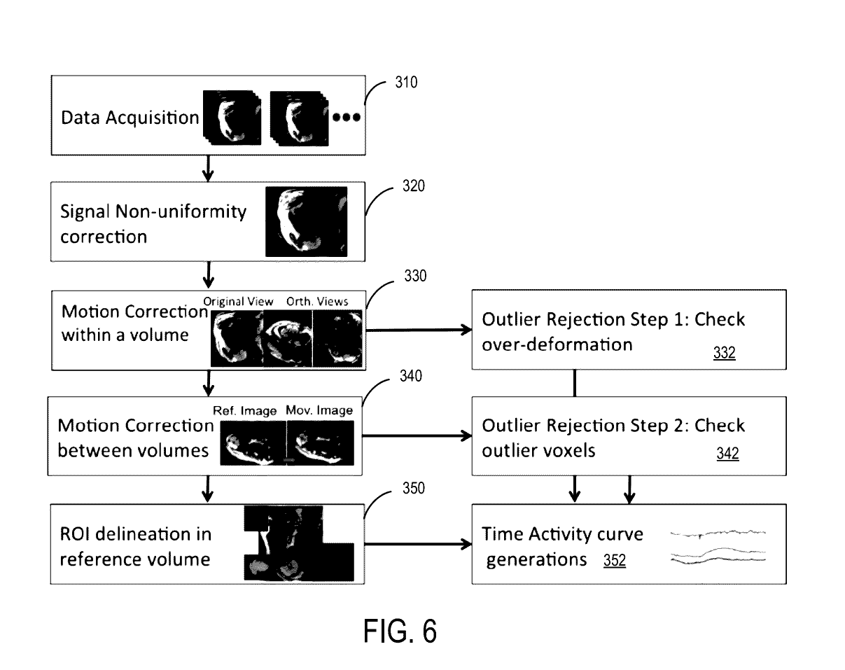

FIG. 6 is a flowchart illustrating steps of an example method to estimate and eliminate signal non-uniformities and motion artifacts in the MRI images according to the disclosure. Here, in step 310, data may be acquired with a single shot gradient echo echo-planar imaging (EPI) with repetition time (TR), for example, between 2 s and 20 s, echo time (TE) 32-36 ms, flip angle (FA) 90°, 3 mm slices acquired in interleaved order, and in-plane resolution of 3×3 mm². The number of slices may be modified to cover the whole uterus and the number of measurements was adjusted for 30 minutes total acquisition time. In one example, scans were performed on a 3T Skyra scanner (Siemens Healthcare, Erlangen, Germany) using an 18-channel body and 12-channel spine receive arrays. Total number of four singletons and six twin pregnancies within 26 and 34 weeks of gestational age were scanned. Informed consent was obtained in all cases. Pregnant women participating in this study were transiently exposed to an increased inspired oxygen concentration. The maternal oxygenation protocol was designed as three consecutive ten minutes episodes: initial normoxic episode (21% O2), hyperoxic episode (100% O2, 15 L=min), and a final normoxic episode (21% O2). The oxygen paradigm was designed in consultation with an anesthesiologist with specialty experience in Obstetrical Anesthesia to ensure the safety of the mother and the fetus. Oxygen was supplied via non-rebreathing facial mask during BOLD acquisition and the facial mask was applied without interfering with the BOLD MRI scan while the pregnant woman remained in the bore magnet. Subjects were lying on their sides in the scanner.

In step 320, the MRI system performs signal non-uniformity correction. In step 330, the MRI system performs motion corrections within a volume. In step 340, the MRI system performs motion corrections between volumes. In step 350, the MRI system delineates ROI in the reference volume. The MRI system may perform two separate outlier rejections steps 332 and 342 respectively after motion correction steps 330 and 340. In step 352, the MRI system may generate a time activity curve.

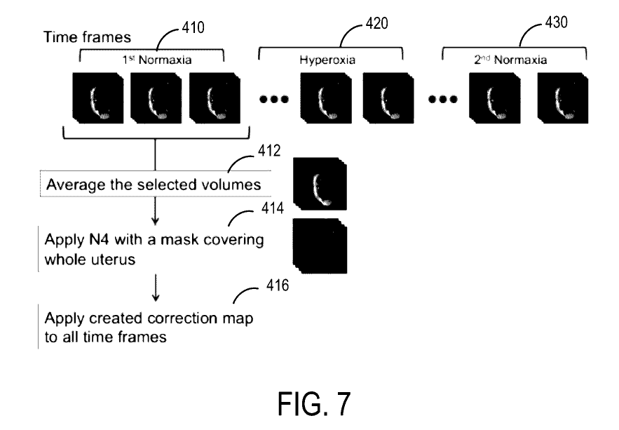

FIG. 7 is a flowchart illustrating steps of an example method using N4 Bias correction on a series of MRI images spanning multiple time frames, including 1st normaxia 410, hyperoxia 420, and 2nd normaxia 430. For signal non-uniformity correction, the MRI system may employ the N4ITK method, available from the 3D Slicer program or other applications, which searches for a smooth multiplicative field that maximizes the high frequency content of the distribution of tissue intensity using a robust B-Spline approximation algorithm. In this algorithm, image is modeled as:

v(x)=u(x)f(x)+n(x) (3).

In the above equation, v is the input image, u is the uncorrupted image to be restored, f is the bias field, and n is the noise, assumed to be Gaussian and independent. Assuming a noise-free scenario and using logarithmic transforms (e.g., ̂u=log u), corrected log image ̂un is constructed by an iterative algorithm that repeatedly applies an update rule:

$${\hat{u}}^{1} = \hat{v} - \underbrace{{S}^{*} \{ \hat{v} - E [ \hat{u} \vert \hat{v} ] \}}_{{\hat{f}}_{r}^{n}} \\ {\hat{u}}^{2} = {\hat{u}}^{1} - \underbrace{{S}^{*} \{ {\hat{v}}^{1} - E [ \hat{u} \vert {\hat{u}}^{1} ] \}}_{{\hat{f}}_{r}^{n}} \\ ⋮ \\ {\hat{u}}^{n} = {\hat{u}}^{n - 1} - \underbrace{{S}^{*} \{ {\hat{u}}^{n - 1} - E [ \hat{u} \vert {\hat{u}}^{n - 1} ] \}}_{{\hat{f}}_{r}^{n}} . \qquad ( 4 )$$

Here, S* is the B-spline approximator to estimate the residual bias field at the nth iteration \hat{f}ᵣⁿ and E[û|ûⁿ⁻¹] is the expected value of the true image given the current estimate of the corrected image such that E[û|ûⁿ⁻¹]=∫_{-∞}^{∞}ûp(û|ûⁿ⁻¹), where p(û|ûⁿ⁻¹) is the conditional probability of û given ûⁿ⁻¹.

To improve robustness, the method estimates a single bias field map from a collection of time frames in resting state (i.e., first 10 minutes) via averaging 412. Since the bias correction algorithm does not have the prior constraints on the intensity changes due to the maternal oxygenation in second and third episodes of the oxygenation paradigm, these volumes were excluded from estimation. In order to quantify the difference between the volumes collected in the first ten minutes, each volume was compared with the rest of the volumes using mean square error difference (MSE). Volumes with high MSE difference compared to other volumes were excluded from averaging.

3D-N4 bias correction method implemented in the advanced normalization tools (ANTs) registration suite may be used. In this case, at 414, a mask may be used to cover part or the whole uterus, and method can be applied within the mask with default B-Spline fitting parameters, while shrink factor was set to 3 and the number of iterations was set to 400. Thereafter, the correction map is applied to the time frames 416. FIG. 7 demonstrates an example application of N4 Bias correction for signal non-uniformity correction in the disclosure.

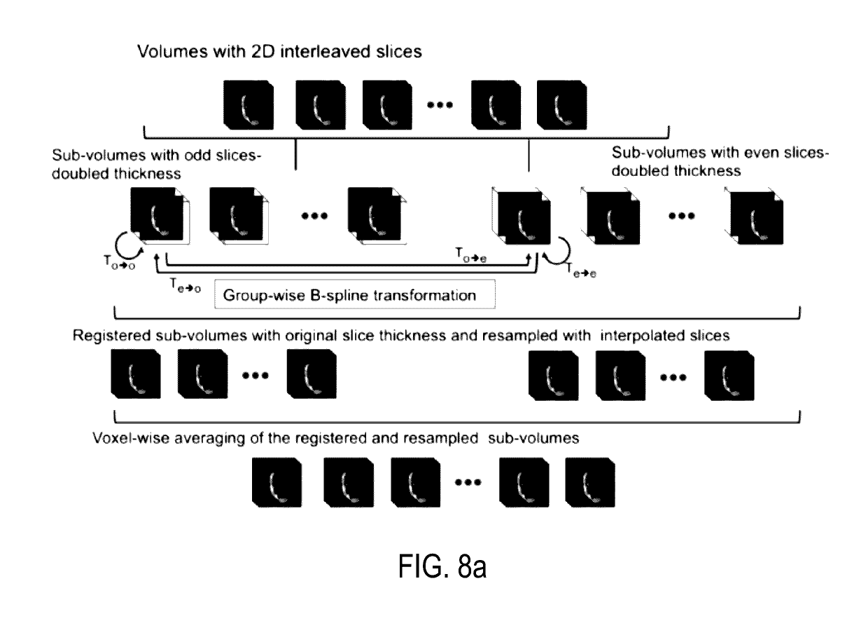

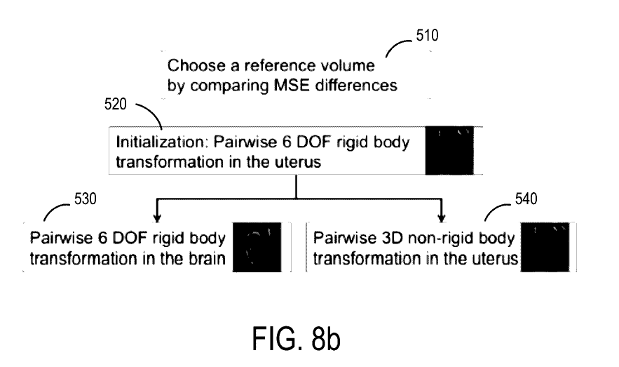

FIG. 8a is a flowchart illustrating steps of motion correction within a volume using group-wise registration. For each time point, volumes were created using 2D slices acquired in interleaved manner and significant effect of motion was observed between consecutive slices. To compensate for motion between even and odd slices, signal non-uniformity corrected volumes were separated into two sub-volumes including only even slices and only odd slices and slice thickness was doubled for each slice to bring the slices into a common coordinate system. FIG. 8a illustrates a method to reduce motion artifacts between the two sub-volumes.

The group-wise method based on multiple pair-wise registrations with a non-rigid body transformation was applied to align two sub-volumes into mid-point image space. In this approach, each sub-volume, Pₑᵥₑₙ (P_{odd}) was taken as a fixed image and two independent transformations were created between P_{odd} and Pₑᵥₑₙ (Pₑᵥₑₙ and P_{odd}) and between Pₑᵥₑₙ and Pₑᵥₑₙ (P_{odd} and P_{odd}). Note that the transformation between Pₑᵥₑₙ and Pₑᵥₑₙ (P_{odd} and P_{odd}) is the identity. Each sub-volume was transformed into an average image space using the inverse of the transformation:

Tₑᵥₑₙ(x)=½(T_{even→odd}(x)+T_{even→even}(x)) and T_{odd}(x)=½(T_{odd→even}(x)+T_{odd→odd}(x)) (5).

After applying inverse transformations, registered sub-volumes were returned back to the original slice thickness and missing slices were filled by linear interpolation. Motion corrected volume was created by the voxel-wise averaging of the registered and resampled sub-volumes.

For a non-rigid transformation, 3D B-spline model was used with three level multi-resolution strategy and a maximum number of 2500 iterations per resolution. Optimization was performed using gradient descent. To choose the similarity metric, mutual information, sum of squared difference and normalized correlation coefficient were tested in a single data set. The results were similar for all three metrics. For the rest of the data sets, mutual information was used, due to its known convenience for aligning images with different intensity distributions, which may be critical for functional data collected during maternal oxygenation paradigm, although the data series were acquired with the same contrast parameters.

The motion correction steps may be implemented using Elastix or other open source image registration software.

FIG. 8b is a flowchart illustrating steps of motion correction between volumes using pair-wise registration. The MRI system may choose a reference volume 510 as a fixed image I_{f}(x) with an image space Ω_{F} while the rest of the volumes were treated as moving images Iₘ(x), with an image space Ω_{M} and registration was performed to create a transformation T: Ω_{F}→_{M} that spatially aligns Iₘ(T(x)) with If (x). To choose the reference volume, MSE difference was computed between each corrected volume and the volume with the least MSE difference with respect to the remaining volumes was chosen as a reference volume.

The masks (i.e. the uterus mask, the fetal brain mask) delineated manually on the average volume (i.e. the average of all time series after intra-volume motion correction) in ITK-SNAP (20) were used for motion correction between volumes. As an initialization 520, six degrees of freedom rigid transformation was defined as a mapping from the reference volume to the moving image within the mask including a whole uterus, i.e., T: Ω_{F}⊂Rᵤₜₑᵣᵤₛ→Ω_{M}⊂Rᵤₜₑᵣᵤₛ.

To compensate for motion in the deformable tissues in a uterus (e.g., placenta and fetal liver), a non-rigid body transformation 540 was computed while using the initial rigid body transformation 530 as an initialization component such that the result of the initial rigid registration T_{R0} was composed with a non-rigid transformation T_{NR} in the way that T(x)=T_{NR}(T_{R0}(x)). In order to avoid excessive deformation in the fetal brain, a second rigid transformation may be estimated as a mapping from the reference volume to the moving image within the mask including the whole brain.

A gradient descent optimization method may be used to maximize the mutual information. Grid size for B-spline transformation and gradient descent optimization parameters may be determined in a subset of the volumes by visual inspection. Customized parameter file was created for each case, separately.

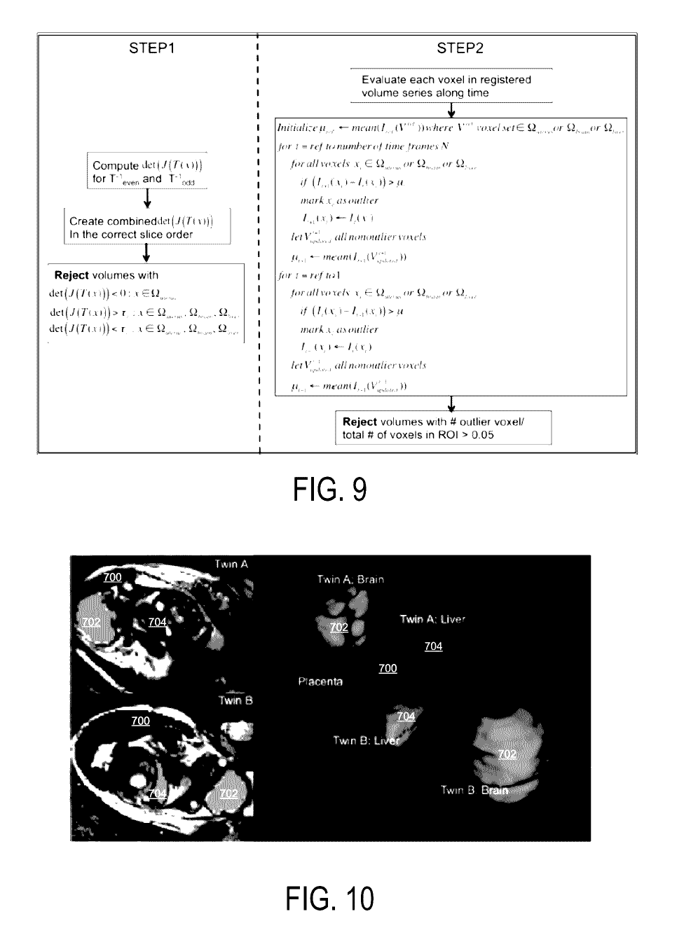

FIG. 9 is a flowchart illustrating steps of outlier rejection after motion correction. Due to the severe motion, the motion correction algorithm may fail for some of the volumes. In order to detect these volumes, the MRI system may adopt a two-step outlier rejection procedure. Since the deformations in fetal organs and placenta are different, the outlier rejection can be performed for each ROI separately. In the first step 610, the deformations within voxels in the ROI were quantified by computing the determinant of the Jacobian of transformations det(J(x)) after motion correction within the volume. The determinant of the spatial Jacobian at any point indicates whether the transformation expands or shrinks space near the given point. Furthermore, a positive determinant means that transformation preserves orientation and a negative determinant corresponds to transformation that reverses orientation. In this first step of the outlier rejection, all volumes that contained voxels with negative determinants of the Jacobian were directly rejected. In order to take into account the differences between the elasticity of different tissues, over-compression and over-expansion in a voxel were evaluated with different thresholds (τ_{c}; τₑ). Volumes that contained voxels with det(J(x))<τ_{c} or det(J(x))>τₑ were also rejected from the dynamic analysis of the ROI. Thresholds τ_{c} and τₑ may be chosen empirically based on a subset of the data.

In the second outlier rejection step 620, the MRI system may evaluate each voxel in the region of interest (ROI) by using the averaged signal intensity μₜ for that ROI at time t and the temporal change of the signal intensity Iₜ₊₁(x)−Iₜ(x). The difference between the intensities of a voxel at time t and the next time point t+1 was compared with the mean signal intensity at time t. When the temporal difference was higher than the mean value, this voxel at time t+1 was marked as an outlier and rejected from the ROI. For the next time point (e.g., time t+2), the intensity of a voxel assigned as an outlier (e.g., at time t+1) was replaced with its value in the previous time point at which it was not assigned as an outlier, and the mean signal intensity was recalculated using the updated ROIs excluding outliers. Volumes with more outlier voxels than 5% of the total number of voxels in the ROI were rejected during region specific dynamic analysis.

FIG. 9 illustrates the outlier rejection procedure. The computations may be carried out in the MATLAB 8.6.0 (The MathWorks, Inc., Natick, Mass.) environment or other software program.

Regions of interest (i.e., placenta, fetal liver and brain) used in temporal analysis were drawn manually in ITK-SNAP using the reference frame. The same mask was used in each registered volume to compute average signal intensities. FIG. 10 illustrates the ROIs (placenta, livers and brains) for a twin case, which shows an example of segmentation of placenta (700), fetal brains (702) and fetal livers (704).

The proposed pipeline was applied to ten volume series and results of each step of the pipeline were visually inspected. For quantitative evaluation, placenta, fetal liver and brain masks were manually drawn in the registered data sets and Dice similarity coefficients (i.e., 2|A∩B|/|A+B| where A and B are automatic and manually delineated regions and |•| denotes voxel count) were computed with respect to the masks delineated in the reference volume and the updated masks after the outlier voxel rejection.

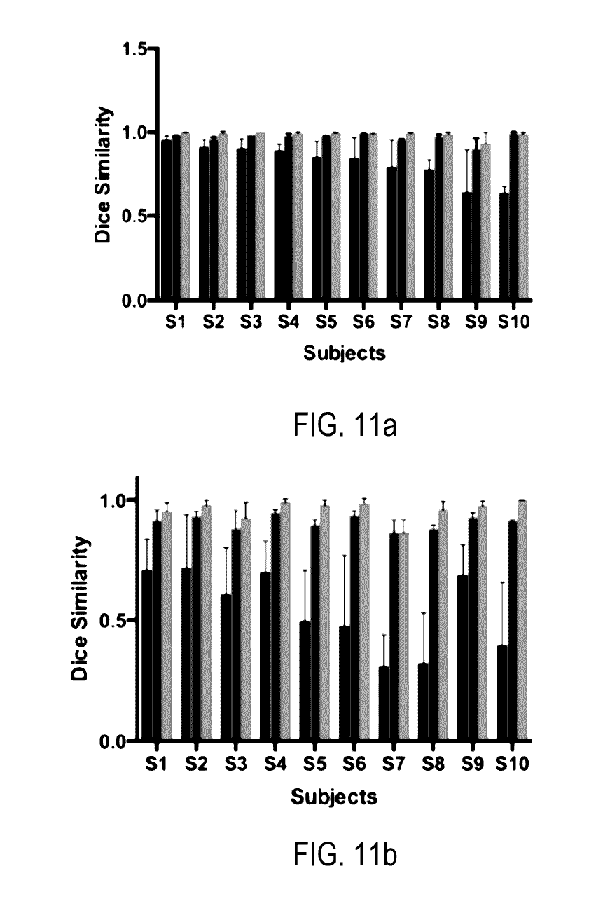

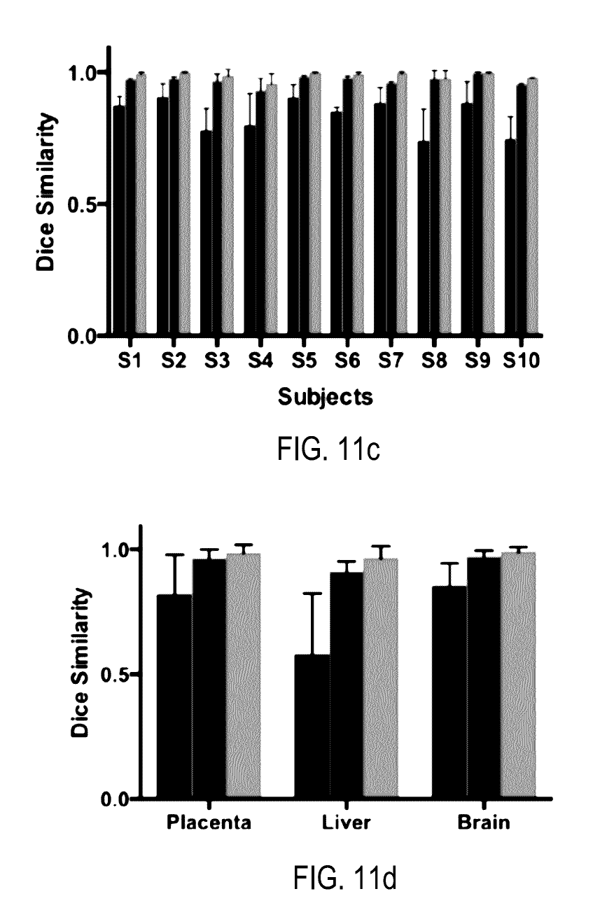

FIG. 11a-c report volume overlap statistics. Moreover, placenta, fetal liver and brain masks were delineated in the original data sets and Dice similarity coefficients were computed with respect to the masks delineated in the reference volume and shown in the same figure. FIG. 11d reports combined Dice coefficients for the placenta, fetal liver and brain. After applying our methods, the mean values of the Dice coefficients improved from 0.82 to 0.96 for the placenta, from 0.58 to 0.91 for the liver, and from 0.85 to 0.97 for the brain. FIG. 11a shows volume overlap computed before applying the pipeline (blue), after applying the pipeline (red), and after outlier voxel rejection (green) for placenta region. FIG. 11b shows volume overlap computed before applying the pipeline (blue), after applying the pipeline (red), and after outlier voxel rejection (green) for liver region. FIG. 11c shows volume overlap computed before applying the pipeline (blue), after applying the pipeline (red), and after outlier voxel rejection (green) for brain region. The error bars are computed across volumes in the time series. FIG. 11d shows comparison of combined Dice coefficients with the error bars computed across all subjects and all volumes for each subject.

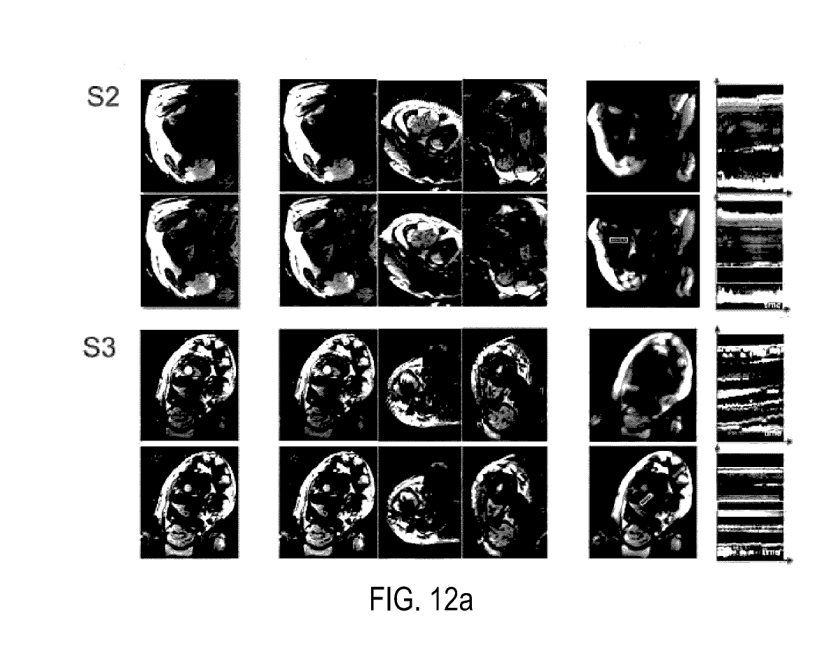

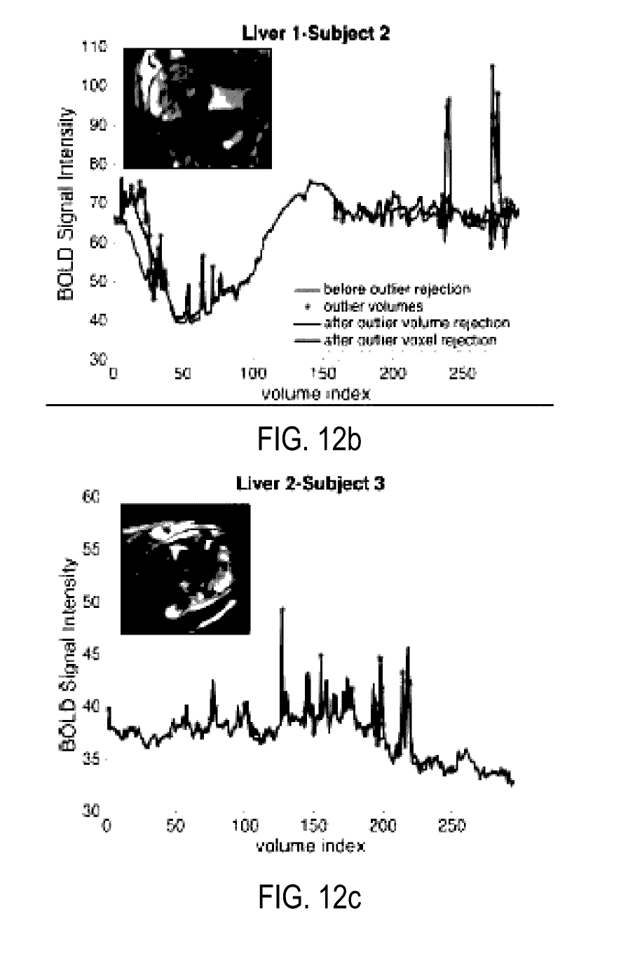

FIGS. 12a-c show the results of each step in the pipeline for two subjects in a study. In FIG. 12a, the bias field correction helps to improve the contrast between different organs and to enhance the boundaries. The first three columns include: Results of signal non-uniformity correction, motion correction within a volume, and motion correction between volumes for two subjects. Upper row (I) left to right shows the original data before bias correction, slice plane view-sagittal (left), orthogonal views-coronal (middle) and axial (right) demonstrating discontinuity of tissue boundaries due to the motion with red arrows, single slice of the average volume, and the intensity profile along the red line shown in the average volume as a function of time. Images in the lower row (II) show the same data after each step of the pipeline.

In FIG. 12a, the second row of the second column for each subject demonstrates that the inter-slice motion artifacts were removed in the orthogonal views with intra-volume motion correction (red arrows highlight the regions with the discontinuity of tissue boundaries due to the motion). The third column shows that sharpness of the placental and fetal regions (i.e. fetal abdomen) in the average volume was improved with the correct alignment of the volumes. Moreover, the fluctuations in intensity profiles shown as a function of time along the red line indicated in the average volumes were decreased, especially in fetal liver and placental regions (highlighted with yellow and blue boxes, respectively). The last column presents signal intensity as a function of the volume index for the fetal livers of two subjects. Sagittal views next to the plots demonstrate the ROIs where outlier voxels were highlighted. Here, voxel-wise outlier rejection improves the signal intensity behavior without the need to eliminate entire volumes, as long as there is no over compression or expansion that disrupts the overall tissue appearance.

FIG. 12a shows intermediate images after each processing step in the pipeline for two subjects. FIG. 12b shows intensity vs. time for the fetal liver of the first subject. FIG. 12c shows intensity vs. time for the fetal liver of the second subject.

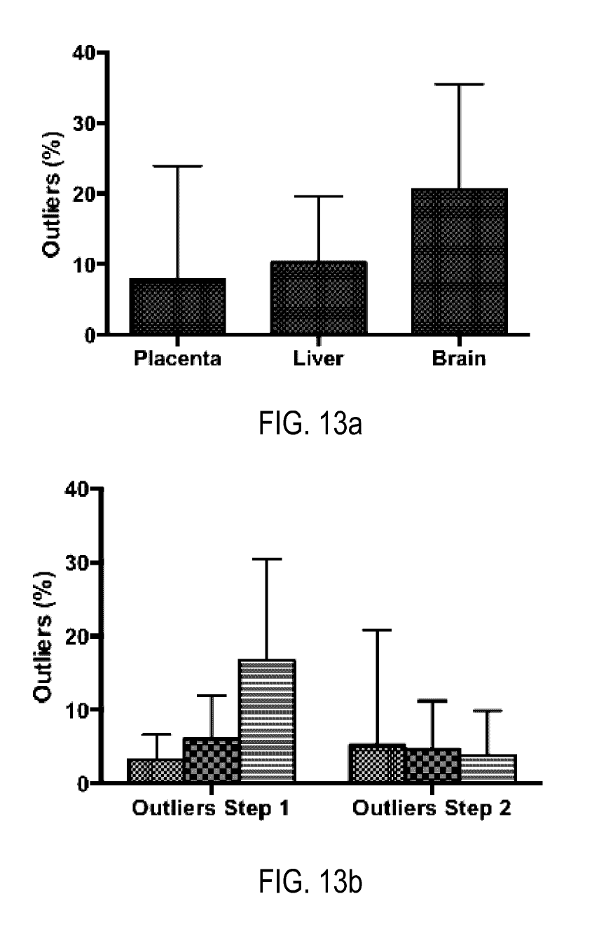

After visual inspection of local expansion and compression, thresholds (τ_{c}; τₑ) for over-compression and over-expansion for the placenta, fetal liver and fetal brain were set as (0.5; 1.5), (0.7; 1.3), (0.8; 1.2) respectively. FIG. 13a reports the overall percentages of the outlier volumes excluded from the analysis. FIG. 13a shows overall percentages of rejected volumes in the analysis of different regions in the fetus.

FIG. 13b shows percentage of rejected volumes in the analyses of the different regions after each step of outlier detection.

In summary, the computational pipeline contains four main steps: signal non-uniformity correction, intra-volume motion correction, inter volumes motion correction and outlier detection.

To create a robust signal non-uniformity map, the MRI system uses an average of selected volumes collected in the first ten minutes. Volumes may be selected based on their MSE differences. The MRI system then applies the same bias field map to all time series with the assumption that the position of the receiver coil with respect to the mother was stable during the scan and change of the loading due to the fetal motion has limited effect on the change of the receiver's sensitivity. If separate bias field correction maps for each volume in a single data set are desired, it may be necessary to model the signal intensity change due to maternal oxygenation in the uterus and integrate such model with the signal non-uniformity correction algorithm. The proposed method does not include correction for geometric artifacts due to BO inhomogeneties. The framework may be extended to include a distortion correction as a part of the pipeline for more accurate dynamic analysis.

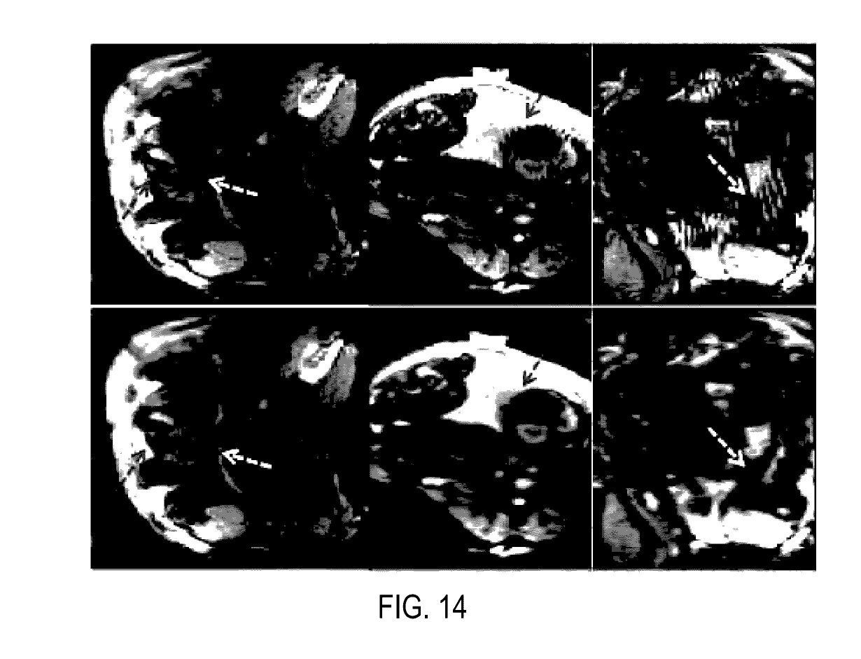

FIG. 14 shows example images illustrating effects of motion correction within the volume to the alignment of the volumes in a reference volume. In this figure, the top row shows sagittal, axial and coronal views of a single volume after alignment to the reference volume without intra-volume motion correction. Red and white arrows indicate the effect of the intra-volume motion on the inter-volumes motion correction in different regions of the uterus. Bottom row indicates the improvement when intra-volume motion corrected images were used for inter volume motion correction. Intra-volume motion was corrected by following an approach based on the non-rigid body registration of sub-volumes formed with even and odd slices, by assuming that fetal and maternal motions are non-trivial between consecutive slices. More sophisticated approaches such as a modified cascaded slice to volume registration for non-rigid body motion can also be investigated in this context.

For both intra-volume motion correction and motion correction between volumes, non-rigid body transformations may be applied to the whole uterus with the parameters adjusted for a deformable tissues without any rigidity masks. In order to eliminate the effect of over-deformation in the brain during intra-volume motion correction, higher thresholds were set for the first step of the outlier detection in brain regions. In FIG. 13b, percentages of the outlier volumes for each ROI were shown after each outlier detection step. Adjusting B-spline transformation parameters for a deformable tissue in intra-volume motion correction and setting lower threshold for brain while evaluating the tissue deformation in the first step of the outlier rejection led to the highest number of outlier volumes in the brain. This fraction can be reduced using different registration methods such that using a rigidity penalty term added to the registration algorithm during intra-volume motion correction. As a result, good alignment for both rigid and deformable tissues can be achieved.

The proposed pre-processing pipeline corrects signal non-uniformities and mitigates fetal and maternal motion in BOLD MRI data sets acquired during oxygen challenge. This method includes signal non-uniformity correction and rigid and non-rigid body motion correction for the whole uterus. Temporal analysis of the fetal and placental responses to different maternal oxygenation episodes is enabled by the proposed method.

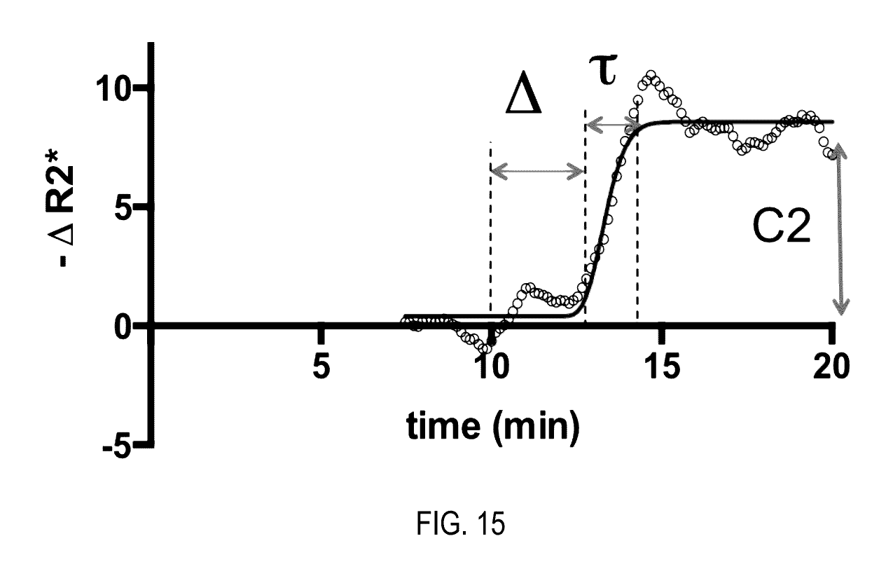

FIG. 15 shows an example signal change curve with two delay parameters to be estimated. The signal change curve may be modeled using the following equation.

R2*(t|α,β,Δ,C₁,C₂)=C₁+C₂·(t−Δ)^{α-1}·e^{-(τ-Δ)/β} [symbol] P_{oxy}(t,Δ) (6).

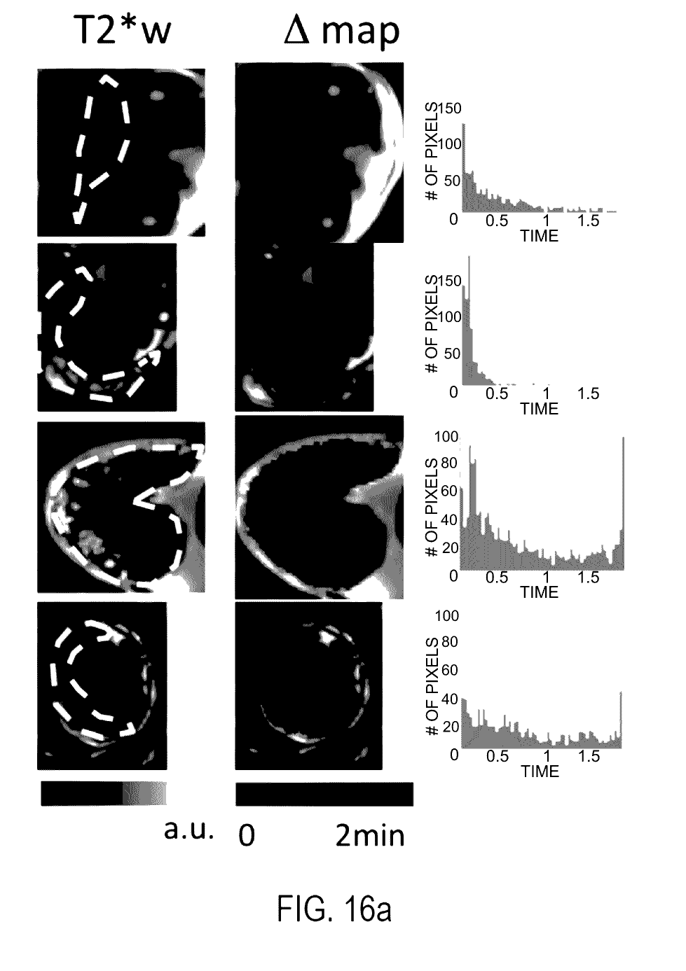

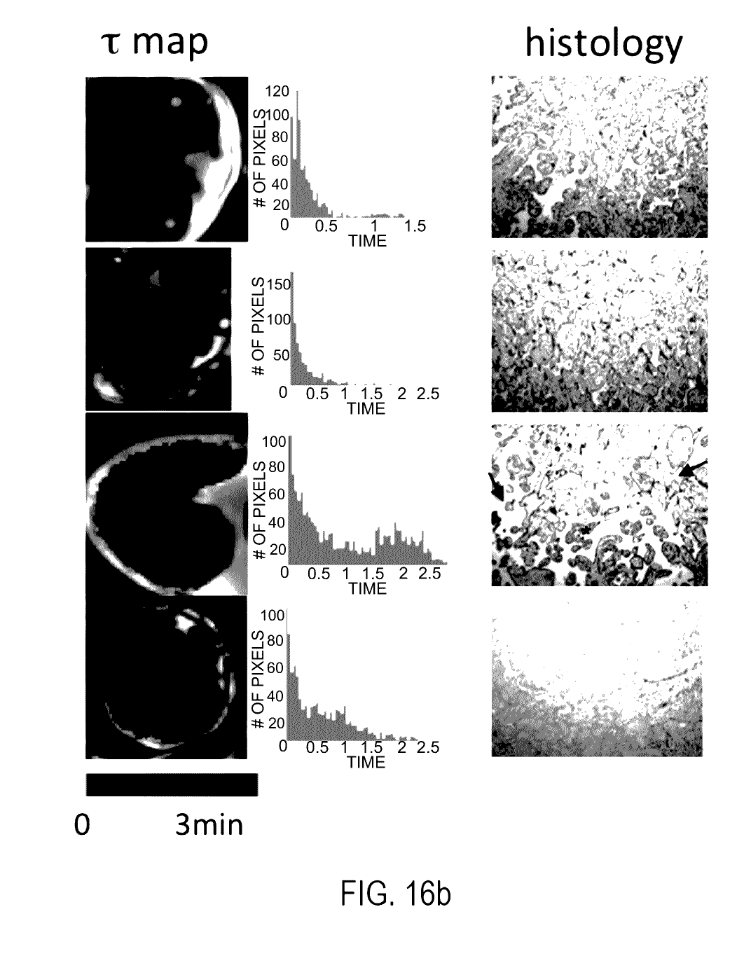

In Equation (6), P_{oxy}(t, Δ)=1 for t≧Δ, and P_{oxy}(t, Δ)=0 for t<Δ. τ=(α−1)·β. The MRI system may include a computer system that fits the obtained BOLD data using the above equation and obtain Δ map and τ map. Examples of the Δ map and τ map are shown in FIGS. 16a-b. FIG. 16a shows examples images illustrating T2* map and an estimated first delay parameter map superimposed on the MRI images. FIG. 16b shows examples images illustrating an estimated second delay parameter map superimposed on the MRI images and histograms.

Here, the proposed method offers 1) a map that characterizes how placenta regions respond to a sudden maternal oxygen increase; 2) % of placental region that respond differently to oxygen exposure; and 3) correlations of oxygen intake placental regions and fetal organs. Using the information post hyperoxia, the oxygen clearance of placenta can be determined after switching oxygen back to room air, a measurement of effective placenta reserve. The method may be used to measure placental function in vivo.

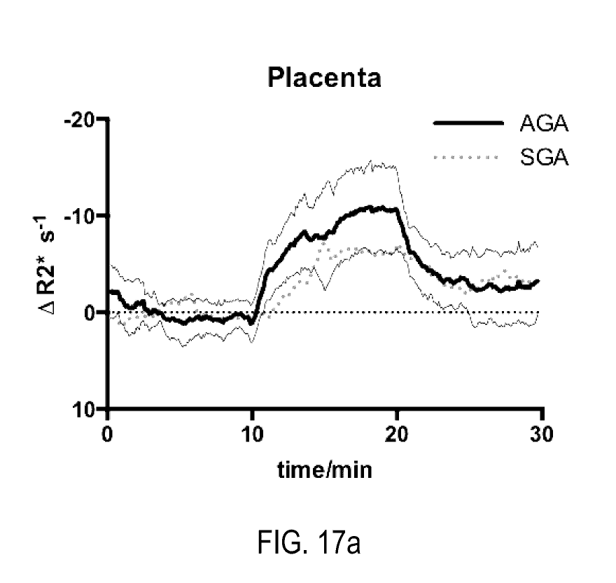

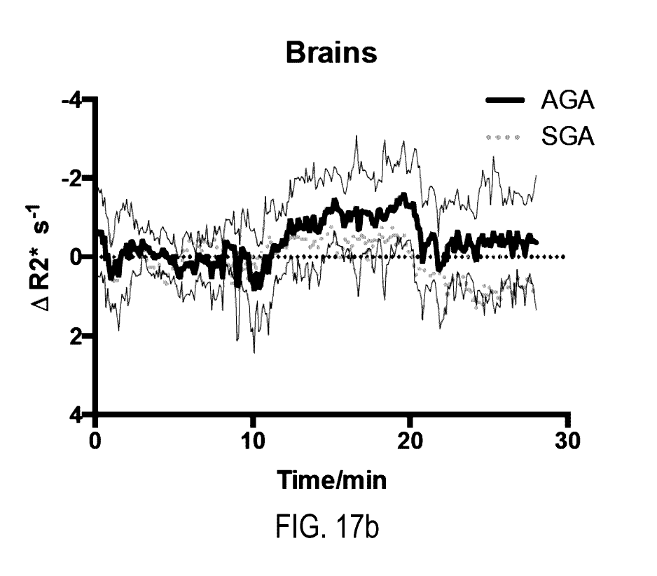

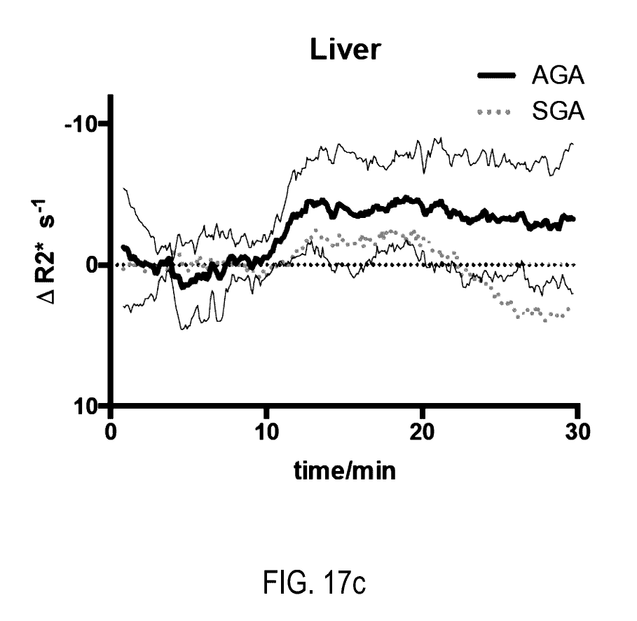

FIG. 17a shows examples illustrating grouped time activity curves of ΔR2* for placenta. Here, the subjects were scanned on a 3T Skyra scanner (Siemens Healthcare, Erlangen, Germany) using an 18-channel body and 12-channel spine-receive arrays. The entire uterus was imaged using ss-GRE-EPI with in plane resolution 3×3 mm2, slice thickness 3 mm, interleaved; TR=5-7 s, TE=30-38 ms, FA=90°, BW=2.3 kHz/px. Total acquisition time 30 min. Maternal oxygen supply was alternated during the scan via non-rebreathing facial mask giving three 10 min episodes: 1. Normoxic (21% O2), 2. Hyperoxic (100% O2, 15 L/min), 3. Normoxic (21% O2).

Bias field was estimated from the first normoxic episode using N4, and applied to all time points. Intra volume motion was corrected using non-rigid group-wise registration; pairwise registration was carried out with organ specific rigid and non-rigid body transformations in Elastix to correct for inter-volume motion. Outlier volumes were excluded based on transformation fields and temporal signal change in each voxel. Manual segmentation of placentae and fetal organs on reference frame was performed and propagated to all time frames. Time activity curves (TAC) were generated by taking mean signal intensity of ROI at each time point, filling outlier time points by linear interpolation. Finally, signal intensities were converted to ΔR2*, according to the following Equation (7), and resampled to give identical temporal resolution before statistical analysis across subjects.

S(t)=A*e−R2*t,ΔR2*=−log(S(TE)/S(TE))/TE, (7)

Here, S (TE) is the average of the first 10 min signal. Since R2* is inversely associated with blood oxygenation level SO2. The decrease of R2* may be used as a marker for increased blood oxygenation.

The long-range temporal correlated data may be modeled using functional data analysis methods. The computer system may calculate mean and standard deviation as functions of time for AGA and SGA groups, and t-statistic and p-value functions comparing the group means. Average rate of signal change and between-group t-statistics at various time intervals was tabulated. Each individual subject was also examined for timing of significant oxygenation level increase at initiation of hyperoxia, at the end of hyperoxia, and at the end of second normoxic episode.

All placenta had significant oxygenation increase. In group comparison between AGA and SGA shown in FIG. 17a, the placenta exhibit slower rate of R2* change in SGA cases vs. control both at the beginning of hyperoxia and post hyperoxia (ΔR2* per minute: −0.78 s⁻¹ min vs.−3.5 s⁻¹ min, p<0.0001, and 0.61 s⁻¹ min vs. 2.3 s⁻¹ min, p<0.01, measured at 11 min and 21 min respectively), indicating less efficient oxygen delivery in SGA placentae.

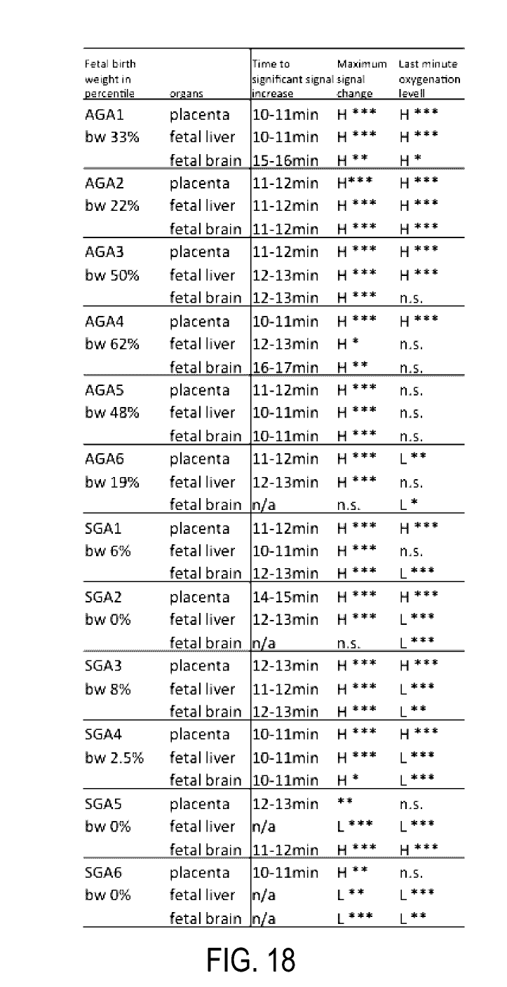

FIG. 17b shows examples illustrating grouped time activity curves of ΔR2* for fetal brains. FIG. 17c shows examples illustrating grouped time activity curves of ΔR2* for fetal livers. In the fetal liver the difference in residual ΔR2* (SGA−control)=6.5 s⁻¹ p<0.01, and ΔR2* (SGA−control)=1.1 s⁻¹ p<0.05 in the fetal brain, which indicate lower oxygenation level for SGA cases in both fetal liver and brain. Further, many SGA fetuses not only have lower oxygenation level compared to AGA fetuses, but they also exhibit lower oxygenation than the baseline (Table 1 in FIG. 18). Different trends between fetal brain and liver responses to hyperoxia are observed in some cases, which suggests variations in fetal hemodynamic auto-regulation. From the results above, BOLD MRI with maternal hyperoxia shows great potential in prenatally differentiating SGA fetus from controls. They have distinct response to maternal hyperoxia exposure. Their placentae show significant difference in rate of oxygen uptake as well.

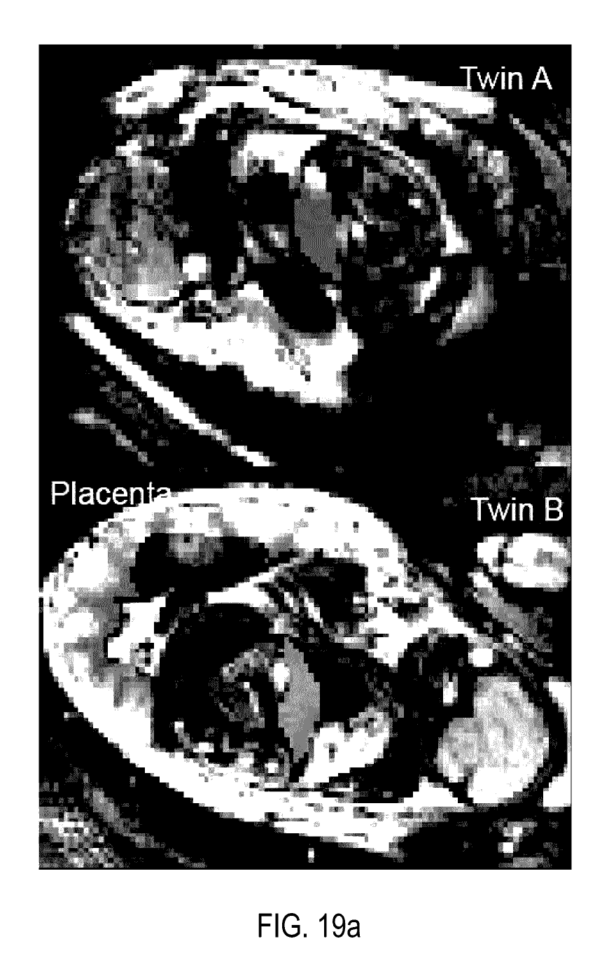

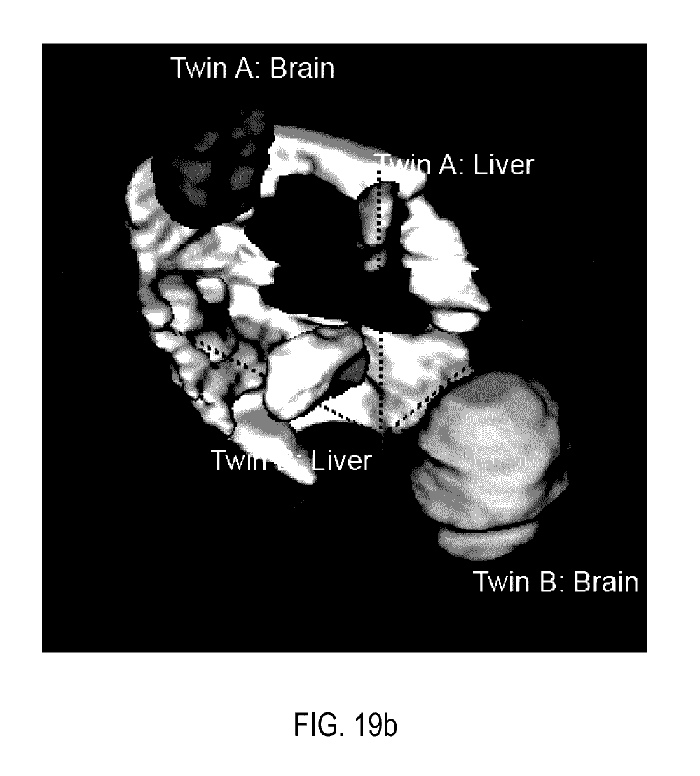

FIG. 19a shows example images illustrating segmentation of placenta and fetal organs in twins separately. FIG. 19b shows example images illustrating segmentation of placenta and fetal organs in twins together. The examples show segmentation of placenta and fetal organs in genetically identical monochorionic twins. Placental regions may be chosen near the cord insertion to avoid contamination under the supervision of an experienced radiologist

The proposed method maps oxygen delivery timing in human placenta. Healthy placentae show Temporal delay (τ) map that agree with normal perfusion timing in response to maternal hyperoxygenation. Pathological placentae exhibit increased delay time and dispersion of oxygen arrival across the placenta.

The present invention has been described in terms of one or more preferred embodiments, and it should be appreciated that many equivalents, alternatives, variations, and modifications, aside from those expressly stated, are possible and within the scope of the invention.

1. A method for generating images using a magnetic resonance imaging (MRI) system, the method comprising:
applying a pulse sequence on the MRI system to obtain a first set of blood oxygenation level dependent (BOLD) MRI images of a pregnant subject during a first time period;
applying the pulse sequence on the MRI system to obtain a second set of BOLD MRI images of the pregnant subject during a second time period;
automatically extracting one or more regions of interest that include a placenta of the pregnant subject in the first and second sets of BOLD MRI images;
obtaining BOLD signal changes in the one or more regions of interest based on the first and second sets of BOLD MRI images; and
generating, based on the BOLD signal changes, a map indicating placental oxygen transport.

2. The method of claim 1, further comprising:
determining whether the one or more regions of interest are abnormal at least partially based on the generated map, wherein one or more regions of interest comprise at least one fetal organ in a fetus of the pregnant subject.

3. The method of claim 1, wherein the pulse sequence comprises a single shot gradient echo echo-planar imaging (EPI) with repetition time (TR) modified between 2 seconds and 20 seconds.

4. The method of claim 1, further comprising:
applying a first and second maternal oxygenation protocols respectively during the first and second time periods, wherein the first maternal oxygenation protocol corresponds to a normoxic episode and the second maternal oxygenation protocol corresponds a hyperoxic episode.

5. The method of claim 1, wherein automatically extracting one or more regions of interest in the first and second sets of BOLD MRI images comprises:
correcting signal non-uniformity using a normalization method that searches for a smooth multiplicative field that maximizes the high frequency content of the distribution of tissue intensity using a robust B-Spline approximation algorithm.

6. The method of claim 1, further comprising performing motion correction within a volume by performing acts comprising:
separating volumes into two sub-volumes including only even slices and only odd slices having doubled slice thickness;
performing registration using group-wise B-Spline transformation;
averaging the registered and re-sampled sub-volumes voxel-wise to reduce motion artifacts between the two sub-volumes.

7. The method of claim 1, further comprising performing motion correction between volumes by performing acts comprising:
computing mean square error (MSE) difference between each corrected volume and selecting the volume with the least MSE difference with respect to the remaining volumes as a reference volume;
defining a six degrees of freedom (6DoF) rigid transformation as a mapping from the reference volume to the moving image within a mask comprising a whole uterus;
computing a non-rigid body transformation using mutual information as a metric while using the 6DoF rigid body transformation as an initialization component; and
estimating a second rigid transformation as a mapping from the reference volume to the moving image within the mask including a fetal brain to avoid excessive deformation in the fetal brain.

8. The method of claim 1, further comprising:
quantifying the deformations within voxels in the ROI by computing the determinant of the Jacobian of transformations det(J(x)) after motion correction within the volume;
rejecting volumes that contained voxels with negative determinants of the Jacobian; and
rejecting volumes that contained voxels with det(J(x)) less than a first threshold or greater than a second threshold.

9. The method of claim 8, further comprising:
evaluating each voxel in the ROI by using an mean signal intensity for the ROI at time t and the temporal change of the signal intensity Iₜ₊₁(x) and Iₜ(x);
comparing a difference between the intensities of a voxel at time t and a next time point t+1 with the mean signal intensity at time t;
when the temporal difference is higher than the mean signal intensity, marking this voxel at time t+1 as an outlier and rejecting the outlier from the ROI;
for time t+2, replacing the intensity of the outlier with a value in the previous time point that is not marked as an outlier; and
recalculating the mean signal intensity using the updated ROIs excluding the outlier.

10. The method of claim 1, further comprising:
converting signal intensities to ΔR2* and re-sampling the ΔR2* to give identical temporal resolution before statistical analysis across subjects;
using a cubic B-Spline basis with knots at two-minute intervals and then calculating mean and standard deviation as functions of time for different groups of subjects; and
obtaining t-statistic and p-value based on the mean and standard deviation of the different groups of subjects.

11. A magnetic resonance imaging (MRI) system, comprising:
a magnet system configured to generate a static magnetic field about at least a placenta of a subject arranged in the MRI system;
at least one gradient coil configured to establish at least one magnetic gradient field with respect to the static magnetic field;
a radio frequency (RF) system configured to deliver excitation pulses to the subject;
a computer system programmed to:
control the at least one gradient coil and the RF system to perform a pulse sequence to obtain a first set of blood oxygenation level dependent (BOLD) MRI images on the subject during a first time period;
control the at least one gradient coil and the RF system to perform the pulse sequence to obtain a second set of BOLD MRI images on the subject during a second time period;
automatically extract one or more regions of interest in the first and second sets of BOLD MRI images;
obtain BOLD signal changes in the one or more regions of interest based on the first and second sets of BOLD MRI images; and
generate, based on the BOLD signal changes, a map indicating placental oxygen transport.

12. The MRI system of claim 10, further comprising:
determining whether the one or more regions of interest are abnormal at least partially based on the generated map, wherein the subject is a pregnant woman having a fetus and the one or more regions of interest comprise at least one fetal organ in the fetus.

13. The MRI system of claim 10, wherein the pulse sequence comprises a single shot gradient echo echo-planar imaging (EPI) with repetition time (TR) modified between 2 seconds and 20 seconds.

14. The MRI system of claim 10, wherein the computer system is further programmed to:
apply a first and second maternal oxygenation protocols respectively during the first and second time periods, wherein the first maternal oxygenation protocol corresponds to a normoxic episode and the second maternal oxygenation protocol corresponds a hyperoxic episode.

15. The MRI system of claim 10, wherein the computer system is further programmed to:
correct signal non-uniformity using a normalization method that searches for a smooth multiplicative field that maximizes the high frequency content of the distribution of tissue intensity using a robust B-Spline approximation algorithm.

16. The MRI system of claim 10, wherein the computer system is further programmed to perform motion correction within a volume by performing acts comprising:
separating volumes into two sub-volumes including only even slices and only odd slices having doubled slice thickness;
perform registration using group-wise B-Spline transformation; and
averaging the registered and re-sampled sub-volumes voxel-wise to reduce motion artifacts between the two sub-volumes.

17. The MRI system of claim 10, wherein the computer system is further programmed to:
compute mean square error (MSE) difference between each corrected volume and selecting the volume with the least MSE difference with respect to the remaining volumes as a reference volume;
define a six degrees of freedom (6DoF) rigid transformation as a mapping from the reference volume to the moving image within a mask comprising a whole uterus;
compute a non-rigid body transformation using mutual information as a metric while using the 6DoF rigid body transformation as an initialization component; and
estimate a second rigid transformation as a mapping from the reference volume to the moving image within the mask including a fetal brain to avoid excessive deformation in the fetal brain.

18. The MRI system of claim 10, wherein the computer system is further programmed to:
quantify the deformations within voxels in the ROI by computing the determinant of the Jacobian of transformations det(J(x)) after motion correction within the volume;
reject volumes that contained voxels with negative determinants of the Jacobian; and
reject volumes that contained voxels with det(J(x)) less than a first threshold or greater than a second threshold.

19. The MRI system of claim 18, wherein the computer system is further programmed to:
evaluate each voxel in the ROI by using an mean signal intensity for the ROI at time t and the temporal change of the signal intensity Iₜ₊₁(x) and Iₜ(x);
compare a difference between the intensities of a voxel at time t and a next time point t+1 with the mean signal intensity at time t;
when the temporal difference is higher than the mean signal intensity, mark this voxel at time t+1 as an outlier and reject the outlier from the ROI;
for time t+2, replace the intensity of the outlier with a value in the previous time point that is not marked as an outlier; and
recalculate the mean signal intensity using the updated ROIs excluding the outlier.

20. The MRI system of claim 10, wherein the computer system is further programmed to:
convert signal intensities to ΔR2* and re-sampling the ΔR2* to give identical temporal resolution before statistical analysis across subjects;
use a cubic B-Spline basis with knots at two-minute intervals and then calculating mean and standard deviation as functions of time for different groups of subjects; and
obtain t-statistic and p-value based on the mean and standard deviation of the different groups of subjects.
